(19) United States
(12) Patent Application Publication
(10) Pub. No.: US 2024/0157152 A1
(43) Pub. Date: May 16, 2024

(54) SYSTEMS FOR MONITORING MULTI-IMPLANTABLE DEVICE SYSTEMS

(71) Applicant: Shiratronics, Inc., Brooklyn Park, MN (US)

(72) Inventors: John Michael Page Knox, Brooklyn Park, MN (US); Alan H. Smythe, White Bear Lake, MN (US)

(21) Appl. No.: 18/388,007

(22) Filed: Nov. 8, 2023

Related U.S. Application Data
(60) Provisional application No. 63/424,705, filed on Nov. 11, 2022.

(51) Int. Cl. A61N 1/36; A61B 5/00; G16H 20/40

(52) U.S. Cl. CPC A61N 1/36192; A61B 5/7264; A61B 5/7435; G16H 20/40; A61B 2560/0204; A61B 2562/08

(57) ABSTRACT

A system may include at least two implantable devices, an external system configured to communicate with each of the at least two implantable devices, and a programmer configured to communicate with the external system. The programmer may include a user interface including a display, and a processor. The processor may be configured to provide on the display implantable device icons corresponding to each of the at least two implantable devices, an external system icon corresponding to the external system, and wireless link representations corresponding to wireless links between the external system and each of the at least two implantable devices. The system may use the wireless links for at least one of communication or power transfer.

### PRIORITY

This application claims the benefit of priority to U.S. Provisional Patent Application Ser. No. 63/424,705, filed Nov. 11, 2022, which is incorporated by reference herein in its entirety.

### TECHNICAL FIELD

This document relates generally to medical systems, and more particularly, but not by way of limitation, to systems, devices, and methods for monitoring and/or programming systems with multiple implantable devices.

### BACKGROUND

Medical devices may include devices configured to deliver a therapy, such as but not limited to an electrical or drug therapy and/or to sense physiological or functional parameters or other health-related data. Medical devices may include external wearable devices and may include implantable devices. For example, a medical device may include implantable devices configured to deliver an electrical therapy. Implantable neurostimulators are an example of implantable electrical therapy devices. A fully head-located implantable peripheral neurostimulation system, having at least two implantable devices, designed for the treatment of chronic head pain is a specific example of a system with more than one implantable device.

Neurostimulation systems may include a rechargeable battery, an antenna coil, and circuitry to control the neurostimulation. The systems may include one or more implantable devices configured to connect with an external unit to perform various functions such as recharging the rechargeable battery, diagnostically evaluating the implantable device(s), and programming the implantable device(s).

Various therapeutic and/or monitoring systems may include multiple devices, which may include external devices such a wearable devices, implantable devices, or various combinations thereof. The monitoring and/or programming of these multiple devices may be rather complex and time consuming. It is desirable to improve the ability of a user to quickly and accurately program or assess device states in multi-device systems.

### SUMMARY

An example (e.g., “Example 1”) of a system may include at least two implantable devices, an external system configured to communicate with each of the at least two implantable devices, and a programmer configured to communicate with the external system. The programmer may include a user interface including a display, and a processor. The processor may be configured to provide on the display implantable device icons corresponding to each of the at least two implantable devices, an external system icon corresponding to the external system, and wireless link representations corresponding to wireless links between the external system and each of the at least two implantable devices. The system may use the wireless links for at least one of communication or power transfer.

In Example 2, the subject matter of Example 1 may optionally be configured such that the wireless representations provide a status indicator for a current status for each of the wireless links.

In Example 3, the subject matter of Example 1 may optionally be configured such that the processor is configured to provide on the display a suggested action to remedy a problem with the wireless status.

In Example 4, the subject matter of any one or more of Examples 1-3 may optionally be configured such that the external system includes a headset, and the headset includes at least two external coils. The external system may be configured to provide wireless links. The wireless links may include a first link between a first one of the external coils and a first one of the implantable devices, and a second link between a second one of the external coils and a second one of the implantable devices. The wireless link representations may provide status indicators for corresponding wireless links between each of the at least two external coils and the at least two implantable devices.

In Example 5, the subject matter of Example 4 may optionally be configured such that the processor is configured to provide on the display a suggestion to align a specific one of the external coils when the status indictor for the wireless link corresponding to the specific one of the coils indicates misalignment.

In Example 6, the subject matter of any one or more of Examples 4-5 may optionally be configured such that the external system includes an external device configured to be connected to the headset via a cable, wherein the processor is configured to provide on the display a cable representation corresponding to a connection of the cable between the headset and the external device, and the processor configured to provide on the display, when a status indicator for the cable representation indicates a failed connection between the headset and the external device, a suggestion to remedy the failed connection.

In Example 7, the subject matter of any one or more of Examples 1-6 may optionally be configured such that the processor is further configured to provide on the display a programmer icon corresponding to the programmer. The communication link representations may include a communication link representation corresponding to a communication link between the programmer icon and the external system icon.

In Example 8, the subject matter of any one or more of Examples 1-7 may optionally be configured such that each of the implantable devices include a rechargeable battery/The processor may further be configured to provide on the display charge state representations for the rechargeable battery in each of the implantable devices.

In Example 9, the subject matter of any one or more of Examples 1-8 may optionally be configured such that each of the at least two implantable devices are configured to be programmed with a stimulation configuration. The processor may be configured to guide a user with acceptable programming inputs when programming the stimulation configuration.

In Example 10, the subject matter of Example 9 may optionally be configured such that the stimulation configuration includes an electrode configuration and an amplitude configuration.

In Example 11, the subject matter of any one or more of Examples 9-10 may optionally be configured such that the processor is configured to guide the user to select at least one anode electrode and at least one cathode electrode.

In Example 12, the subject matter of any one or more of Examples 9-11 may optionally be configured such that the processor is configured to guide the user to select allowable amplitudes for a selected electrode configuration.

An example (e.g., “Example 13”) of a system may include at least two implantable devices, an external device and a programmer. Each of the at least two implantable devices may be configured to deliver stimulation to different regions of a body according to different stimulation configurations. The external system may be configured to communicate with each of the at least two implantable devices. The programmer may be configured to communicate with the external system, and configured for use to program the at least two implantable devices by associating the different stimulation configurations with the different regions of the body, and receiving programming inputs for the different stimulation configurations associated with the different regions of the body. The programmer may include a user interface, including a display that includes identifiers for each of the different regions of the body and includes the different stimulation configurations associated with the different regions.

In Example 14, the subject matter of Example 13 may optionally be configured such that the different regions include a left orbital region, a right orbital region, a left occipital region and a right occipital region.

In Example 15, the subject matter of any one or more of Examples 13-14 may optionally be configured such that the display includes, for each of the different regions, an electrode configuration, an amplitude, a pulse width and a frequency.

An example (e.g., “Example 16”) of a method may include optically scanning a code associated with a medical device, wherein a cryptographic key for the medical device is encoded within the code, decoding the code to determine the cryptographic key, establishing encrypted communication with the medical device using the cryptographic key.

In Example 17, the subject matter of Example 16 may optionally be configured such that the code is a two-dimensional barcode for encoding alphanumeric information, and the alphanumeric information includes the cryptographic key.

In Example 18, the subject matter of Example 17 may optionally be configured such that the code is a Quick Response (QR) code.

In Example 19, the subject matter of any one or more of Examples 13-18 may optionally be configured such that a first device performs the optical scanning and the decoding of the code to determine the cryptographic key, the first device uses the cryptographic key to enable encrypted communication using the cryptographic key to encode a first string of bytes to provide an encoded first string of bytes, and sending the encoded first string of bytes to the medical device. The medical device may use a first private key stored in the medical device to decode the encoded first string of bytes to provide a decoded first string of bytes, and may use the decoded first string of bytes to encode a second string of byte to provide an encoded second string of bytes, and may send the encoded second string of bytes to the first device. Both the first device and the medical device may generate session keys based on the encoded first string of bytes and the encoded second string of bytes, and use the generated session keys to provide the encrypted communication.

In Example 20, the subject matter of any one or more of Examples 13-19 may optionally be configured such that the optically scanning the code includes using a camera in a first device to optically scan the code, the decoding the code includes using the first device to decode the code, and the establishing encrypted communication includes establishing encrypted communication between the first device and the medical device.

In Example 21, the subject matter of any one or more of Examples 13-20 may optionally be configured such that a programmer is used to establish encrypted communication with the medical device. The medical device may include an implantable stimulator, an external trial stimulator or a charger.

An example (e.g., “Example 22”) of a method may be performed in a system having at least two implantable devices, an external system configured to communicate with each of the at least two implantable devices, and a programmer configured to communicate with the external system. The method may include displaying a representation of the system on the display, and performing at least one of: determining a link status for at least links between the external system and the implantable devices, displaying the link status within the displayed representation of the system; or determining a status of rechargeable batteries, and displaying the rechargeable battery status within the displayed representation of the system.

In Example 23, the subject matter of Example 22 may optionally be configured to further include suggesting remedial action for the determined link status or the determined status of rechargeable batteries.

In Example 24, the subject matter of any one or more of Examples 22-23 may optionally be configured to further include guiding selection of an allowable stimulation configuration for the system.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate a system that includes implantable device(s) and an external device configured for use to communicate with and charge the implantable device(s).

FIG. 2A depicts two implanted devices with leads to cover both sides of the head with one on the left side of the head and the other on the right side of the head, and FIG. 2B illustrates a charging/communication headset disposed about the cranium.

FIG. 3 illustrates, by way of example and not limitation, a system with at least two medical devices, an external system, and a programmer.

FIG. 4 illustrates, by way of example and not limitation, a process for providing real time updates for link status and/or battery charge status.

FIG. 5 illustrates, by way of example and not limitation, a process for guiding user-selection of an allowable stimulation configuration.

FIG. 6 illustrates, by way of example and not limitation, another process for guiding user-selection of an allowable stimulation configuration.

FIG. 7 illustrates, by way of example and not limitation, a display with representations for a multi-device neuromodulation system.

FIG. 8 illustrates, by way of example and not limitation, by way of example and not limitation, representations in a display of FIG. 7, indicative of a poor link between the headset icon and the right implantable device icon.

FIG. 9 illustrates, by way of example and not limitation, representations in a display of FIG. 7, indicative of a poor link between devices in the external system.

FIG. 10 illustrates, by way of example and not limitation, representations in a display indicative of an allowable electrode configuration.

FIG. 11 illustrates, by way of example and not limitation, representations in a display indicative of an allowable waveform configuration for selectable electrode configurations.

FIG. 12 illustrates, by way of example and not limitation, representations in a display indicative of different regions in the body.

FIG. 13 illustrates, by way of example and not limitation, a screen display for editing a user-selected program in FIG. 12.

FIG. 14 illustrates, by way of example and not limitation, a method for using an optically-scanned code to establish encrypted communication.

FIG. 15 illustrates, by way of example and not limitation, a display of a device used to obtain a cryptographic key for use to establish encrypted communication.

FIGS. 16A-16C illustrate, by way of example and not limitation, a QR code on an implantable device, a patient programmer and an external trial stimulator for use in obtaining a cryptographic key to establish encrypted communication.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various therapeutic and/or monitoring systems may include multiple devices, which may include external devices such a wearable devices, implantable devices, or various combinations thereof. Those of ordinary skill in the art, who have read and understood this document, will understand how to implement the present subject matter with different therapeutic and/or monitoring systems. This document discusses, by way of a specific example, a system with at least two neurostimulation devices. By way of example and not limitation, a neurostimulation system for delivering therapy to treat head pain may use at least two implantable devices (e.g., see U.S. Pat. No. 9,884,190 entitled “Surgical Method For Implantable Head Mounted Neurostimulation System for Head Pain,” U.S. Pat. No. 9,498,635 entitled “Implantable Head Located Radiofrequency Coupled Neurostimulation System for Head Pain,” U.S. Pat. No. 10,258,805 entitled “Surgical Method for Implantable Head Mounted Neurostimulation System for Head Pain,” and U.S. Pat. No. 10,960,215 entitled “Low Profile Head-Located Neurostimulator and Method of Fabrication,” which are herein incorporated by reference in their entirety).

In order for a clinician to program a system with more than one active implantable neurostimulator, the clinician should understand the specific clinical needs of a patient, determine the therapy configuration that would best accommodate those needs, determine the device level settings for each of the devices that would deliver the desired therapy, and interpret the current device status for each of the devices. This becomes a complex process of understanding not only the clinical mechanisms of the therapy but also the device-specific features. For example, the devices may provide status information and may employ specific interactive limits on what can be programmed.

A system with a single neurostimulator may provide feedback through a series of messages with dialog boxes that focus on the particular device that is implanted. However, the use of such dialog boxes would be unduly cumbersome for systems with more than one device.

Various embodiments of the present subject matter allow a system, with multiple devices, to be programmed to deliver the appropriate therapy. A user may be provided with real-time information regarding each device status using an appropriate level of abstraction so the user can quickly assess device information and appropriately program all devices. Real-time feedback may be provided with specific data that accounts for the complexity of a multi-device configuration to allow a user to appropriately program around issues. Thus, a user is provided with the tools to quickly and accurately program or make assessments on current device state in a multi-device system. As used herein, the term real-time indicates quick acquisition and processing of feedback after the occurrence of an event, such that a displayed status indicator is representative of a current status for a user of the system. It is understood that real time may have some processing and transmission delays, and may also be referred to as near real time. Therefore, real-time is understood to be preferably less than a few seconds (e.g., less than 10 seconds, less than 5 seconds, or less than 1 second after the occurrence of the feedback event), depending on the specifics of the system used to acquire and process the data. The real-time updates allow the user to quickly review the effect of any changes (e.g., whether a user-inputted change is effective in improving the functionality of the system).

Various embodiments provide a system that provides a real-time display of multiple devices and programming information of multiple devices in a clinical abstraction. For example, a patient's frame of reference (e.g., map of quadrants in the head), may be used to program the system, such as a multi-device system. By way of example and not limitation, the system may be programmed and/or monitored using references to body locations such as a map of quadrants in the head. Additionally, or alternatively, anatomical landmarks, such as nerves, muscles, bones, joints, or other body parts, may be used to program and/or monitor the system. The system may provide real-time status information for communication links for use in detecting potential issues, and for use in identifying communication errors and identifying subsequent steps to correct errors. A dashboard of real-time information may be displayed to provide real-time information on each of the devices in the system. For example, the dashboard may simultaneously display real-time information for each of the devices in the system. The system may identify errors or possible errors, and provide specific notifications and/or corrective actions to correct or otherwise address. These enhancements allow an improved user experience and simplifies the configuration of therapy for a particular patient.

Various embodiments update a user interface (e.g., a display of a programmer, remote, phone or tablet) in real time by combining user-input data with system state data. The device state data for individual devices may be determined and synthesized to provide the system state data. By the real time analysis, this state and input data, the present subject matter is able to provide the user with situationally-appropriate advice and feedback for utilizing the system. As the user manipulates the system into new states, the system may be configured to quickly and automatically update the advice and feedback for the new states.

Because the present subject matter is updated in real time and presents system state data using various levels of abstraction, the present subject matter allows knowledgeable users to interact uninterrupted (e.g., without having to dismiss modal dialog boxes, or scroll through lengthy instructions), while still providing novice users the feedback they need to achieve their goals. Furthermore, situations requiring user attention can automatically hide once the situation has been resolved. Also, because the present subject matter processes and updates the user interface in response to asynchronous data from peripheral devices, the present subject matter can proactively provide guidance instead of only responding to a user action with an error.

By way of example, this disclosure discusses a fully head located implantable peripheral neurostimulation system designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias. The system may include two implantable devices bilaterally implanted on the right and left sides of the patient's head. However, the present subject matter is not limited to such systems, as those of ordinary skill in the art would understand, upon reading and comprehending this disclosure, how to implement the teachings herein with other systems such as, but not limited to, two or more medical devices that are implantable or wearable.

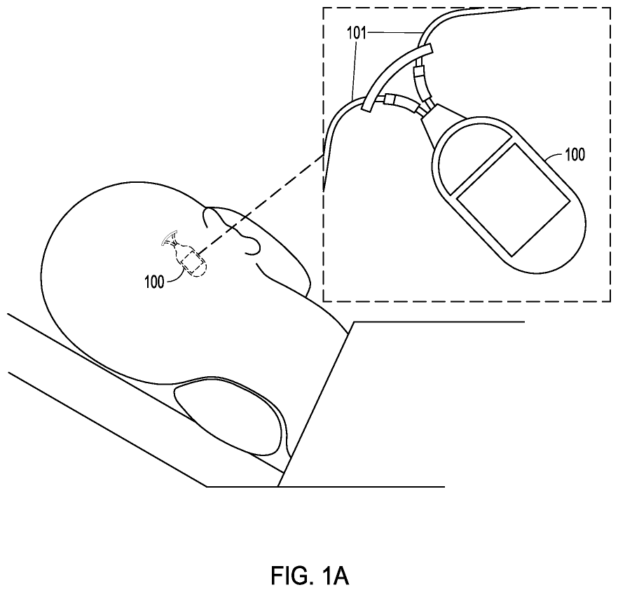

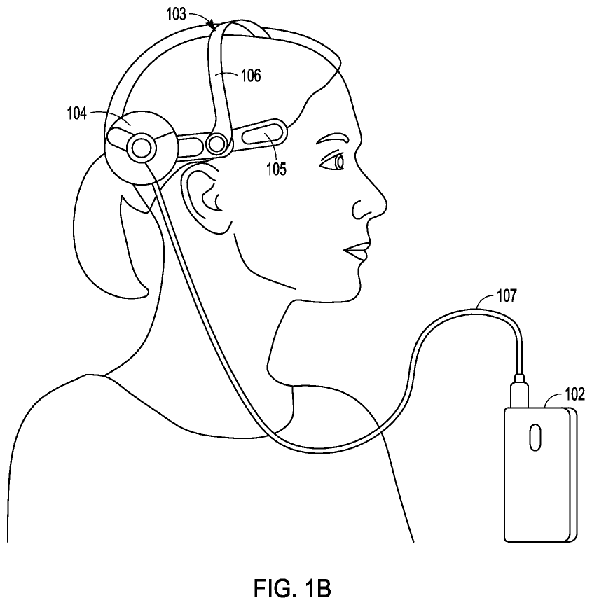

FIGS. 1A-1B illustrate a system that includes implantable device(s) and an external device configured for use to communicate with and charge the implantable device(s). FIG. 1A illustrates an implantable device 100 implanted beneath the skin and over a patient's cranium. The device 100 is illustrated as being implanted behind and above the ear. The implantable device may include one or more leads 101 that may be subcutaneously tunneled to a desired neural target. Each lead may include one or more electrodes. The number of electrodes and spacing may be such as to provide therapeutic stimulation over any one or any combination of the supraorbital, parietal, and occipital region substantially simultaneously. The implantable device 100 may be configured to independently control each electrode to determine whether the electrode will be inactive or configured as a cathode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 1B illustrates an external device 102 and headset 103 configured for use to communicate with and/or charge the implantable device(s) 100. The headset 103 may include an external coil (also referred to as a transmit or Tx coil) 104, and the headset 103 may be configured to position the external coil over an implantable device. For example, the headset 103 may include an adjustable frame 105 on each side of the head that can rotate about a point on a main headset frame 106, and may be configured to provide addition degrees of motion (e.g., sliding or pivoting motion) with respect to the main headset frame 106. These adjustable frames may be used to position the external coils 104 over the implantable devices 100 when the main headset frame 106 is worn. The external device 102 may be electrically connected to the external coil 103 via a cable 107. In some embodiments, the external device 102 may be wirelessly connected to the headset 103. The headset may be configured to wirelessly receive power from the external device and to transfer power from the external coil to the implanted device(s).

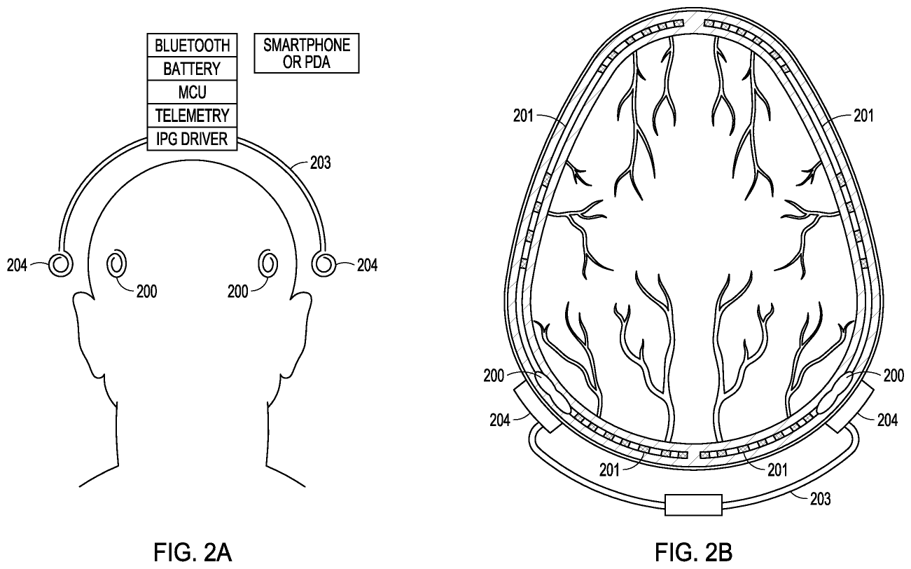

FIG. 2A depicts two implanted devices 200 with leads 201 to cover both sides of the head with one on the left side of the head and the other on the right side of the head, and FIG. 2B illustrates a charging/communication headset 203 disposed about the cranium. The headset 203 may include right and left coupling coil enclosures, respectively that contain coils for coupling to the respective coils in the implants. The coil enclosures interface with a main charger/processor body which contains processor circuitry and batteries for both charging the internal battery in the implantable devices 200 and also communicating with the implanted devices. Thus, in operation, when a patient desires to charge their implanted devices 200, all that is necessary for some embodiments is to place the headset about the cranium with the coils 204 in close proximity to the respective implanted devices 200. In some embodiments, such placement may automatically initiate charging; whereas in other embodiments, the user may initiate charging using an external device. When the headset 203 is worn by a patient, the headset coils (transmit or Tx coils) 204 are placed in proximity to the corresponding receive coil in each respective body-implanted implantable device 200. As illustrated, the headset 203 may include an implantable device driver, telemetry circuitry, a controller or MCU, a battery, and a Bluetooth wireless interface. The headset 203 may also communicate with a personal device such as a smartphone or tablet (e.g., via the Bluetooth interface), for monitoring and/or programming operation of the two implantable devices.

The implantable device may include a rechargeable battery, an antenna (e.g., coil), and an ASIC, along with the necessary internal wire connections amongst these related components, as well as to the incoming lead internal wires. These individual components may be encased in a can made of a medical grade metal, which may be encased by plastic cover. The battery may be connected to the ASIC via a connection that is flexible. The overall enclosure for the battery, antenna and ASIC may have a very low flat profile with two lobes, one lobe for housing the ASIC and one lobe for housing the battery. The antenna may be housed in either of the lobes or in both lobes. The flat, low profile for the housing is beneficial for subcutaneous implantation, as it tends to be more comfortable and cosmetically pleasing for the patient. The use of the two lobes and the flexible connection between the ASIC and the battery allows the implanted device to conform to the shape of the human cranium when subcutaneously implanted without securing such to any underlying structure with an external fixator. However, the implanted device may be anchored in place via suturing or some other anchoring mechanism.

The ASIC and lead may be configured to independently drive the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular programs to provide the necessary therapeutic stimulation. The ASIC may have memory and be configured for communication and for charge control when charging a battery. Each of the set of wires and interface with the ASIC such that the ASIC individually controls each of the wires in the particular bundle of wires. Thus, each electrode may be individually controlled. Each electrode may be individually turned off, or as noted above, each electrode can be designated as an anode or a cathode. During a charging operation, the implanted device is interfaced with an external charging unit via the antenna (e.g., coil) which is coupled to a similar antenna (e.g., coil) in the external charging unit. Power management involves controlling the amount of charge delivered to the battery, the charging rate thereof and protecting the battery from being overcharged.

The ASIC may be capable of communicating with an external unit, typically part of the external charging unit, to exchange information. Thus, configuration information can be downloaded to the ASIC and status information can be retrieved. Although not illustrated herein, a headset or the like may be provided for such external charging/communication operation.

As provided above, FIGS. 1A-2B illustrate a fully head located implantable peripheral neurostimulation system designed for the treatment of chronic head pain. However, the present subject matter is not limited to such systems, as it may be implemented with multi-device systems. The multiple devices in such systems may include one or more implantable devices, or one or more wearable devices. Any of the multiple devices may be configured to deliver a therapy, to monitor health-related parameter(s), or to both deliver a therapy and monitor health-related parameter(s).

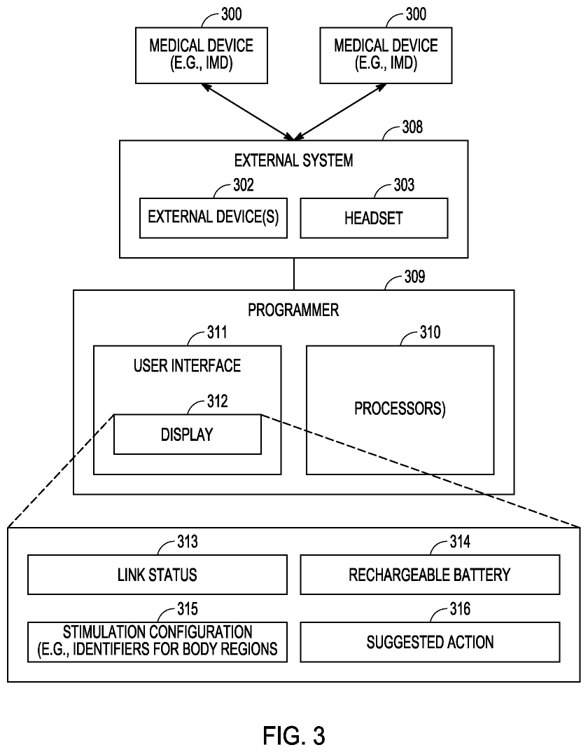

FIG. 3 illustrates, by way of example and not limitation, a system according to various embodiments or the present subject matter. The illustrated system includes at least two medical devices 300, an external system 308, and a programmer 309. The external system 308 may include multiple devices configured to work together to program and/or power the medical devices 300. By way of example, and not limitation, the external system 308 may include external device(s) 302 and a headset 303. For example, the external device(s) 302 may include processing circuitry and power for the headset 308. The programmer 309, the functions of which may be incorporated into one device or distributed over more than one device, includes one or more processors 310 and a user interface 311. The user interface 311 may include a display 312. The processor(s) may operate on instructions to provide information on the display 312. Examples of such information includes one or more of a link status 313, a battery status 314 (e.g., rechargeable or primary battery status indicative of charge or life remaining), a stimulation configuration 315, and suggested actions 316. The link status 313 may provide real-time information concerning the status of a link between devices, such as a wireless link used for communication and/or power transfer. The stimulation configuration 315 may include a user-selectable electrode configuration (e.g., active electrodes configured as anode(s) and cathode(s)) and a user-selectable stimulation waveform configuration (e.g., user-selectable amplitude, frequency, and/or pulse width). The suggested action 316 may include corrective action to avoid potential problems with the links, the battery, or stimulation configuration.

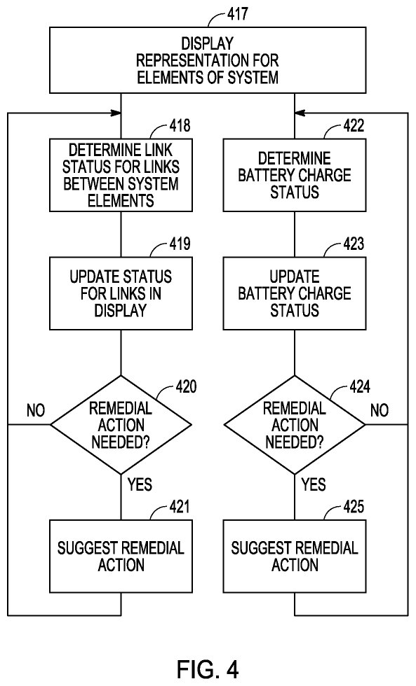

FIG. 4 illustrates, by way of example and not limitation, a process for providing real time updates for link status and/or battery charge status. The process may be performed using the processor(s) within the system, such as but not limited to processor(s) in a programmer. The system (e.g., programmer) has a display on which a representation for elements of the system may be displayed 417. The system elements may include elements of the system illustrated in FIG. 3, such as at least two medical devices, an external system, and a programmer. At 418, the system (e.g., processor(s)) determines the link status between system elements (e.g., a link between the programmer and the external system, and links between the external system and each of the medical devices). The status of the links in the display are updated at 419. For example, if a coil is out of alignment with the implant, the system may detect this misalignment and update the status of that link to show no connection. At 420, the system (e.g., processor(s)) determines if remedial action is needed. If remedial action is needed, the processor(s) suggests the remedial action on the display 421, and the process returns to 418; and if remedial action is not needed, the process returns to 418. This process may be performed in real time to quickly update the displayed link status with a current status of the link, and to provide suggested actions when warranted. At 422, the system (e.g., processor(s)) determines the battery charge status of elements within the system (e.g., the status of batteries, such as rechargeable batteries, used in the medical devices or external system). The battery charge status is updated at 423. At 424, the system (e.g., processor(s)) determines if remedial action is needed. If remedial action is needed, the processor(s) suggest the remedial action on the display 425; and if remedial action is not needed, the process returns to 422. The displayed representation may include representations for both the link status and the battery charge status on the same display screen, or may use different windows for the link status and for the battery charge status. The system may determine the remedial action that is needed. By way of example and not limitation, the system may indicate to the user how to remedy a situation when a cable is not plugged in properly by suggesting the cable to make the connection, and where the cable it needs to be plugged in (e.g., headset cable to charger). Upon correction the real time information to user is immediately updated to notify the user the issue has been corrected. However, any new errors that are identified may be displayed. For example, if a communication or recharge coil is out of alignment it will inform user which coil (s) is/are out of alignment. The suggested remedial action may be a suggestion to the user to adjust the coil such that is in close proximity the implantable device. Information is updated in real time so the user is aware of current status and immediately becomes aware when corrections have been made that addresses a particular issue. By way of example, the system may determine if any of the devices has a low battery indicator, which indicates that the device requires charging. If the battery charge is not sufficient to continue use, the system may notify the user to charge a particular battery before proceeding and will be prevented to continue without sufficient charge of a particular battery. Once the device(s) achieve sufficient charge level they may continue on. The system can instruct the user how to charge their device (e.g., either external devices or implantable device(s)) and provide sufficient information to user on what to do.

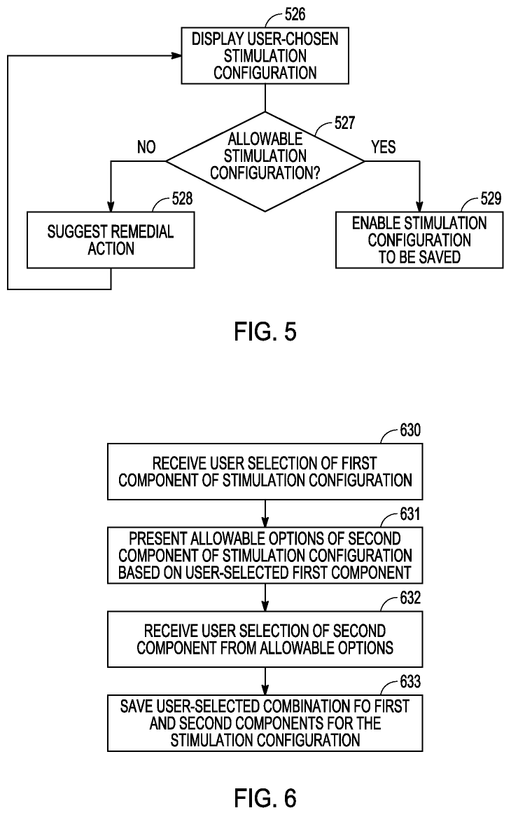

FIG. 5 illustrates, by way of example and not limitation, a process for guiding user-selection of an allowable stimulation configuration. At 526, a user-chosen stimulation is presented on a display. The processor(s) within the system may determine whether the user-chosen stimulation configuration is allowable. If the user-chosen stimulation configuration is not allowable, the process may proceed to 528 where the processor(s) suggest remedial action to a user using the display. For example, the remedial action may be to make a selection of an anode or cathode, or may be to change an amplitude. If the user-chosen stimulation configuration is allowable at 527, then the process may proceed to 529 where the system enables the stimulation configuration to be saved.

FIG. 6 illustrates, by way of example and not limitation, another process for guiding user-selection of an allowable stimulation configuration. Rather than suggesting a corrective action after the user selects a configuration that is not allowable as illustrated in FIG. 5, FIG. 6 guides the selection by providing options so that only allowable stimulation may be selected. At 630, a user selection of a first component of a stimulation configurated is received. The first component may be an electrode configuration (e.g., selection of electrodes to be configured as anode(s) and cathode(s)), or may be a parameter of a stimulation waveform configuration (e.g., amplitude). At 631, the system presents allowable options of a second component of the stimulation configuration based on the user-selected first component. Thus, for example, if the first component is an electrode configuration, the second component may be amplitude (or another waveform parameter). The system only allows amplitudes to be selected that are allowable for the electrode configuration. And if the first component is a waveform parameter such as amplitude, the second component may be another waveform parameter or an electrode configuration. Only allowable combinations are available for user selection of the second component. At 632, the system received the user selection of the second component from the allowable options. At 633, the system enables the user-selected combination of the first and second components for the stimulation configuration to be saved.

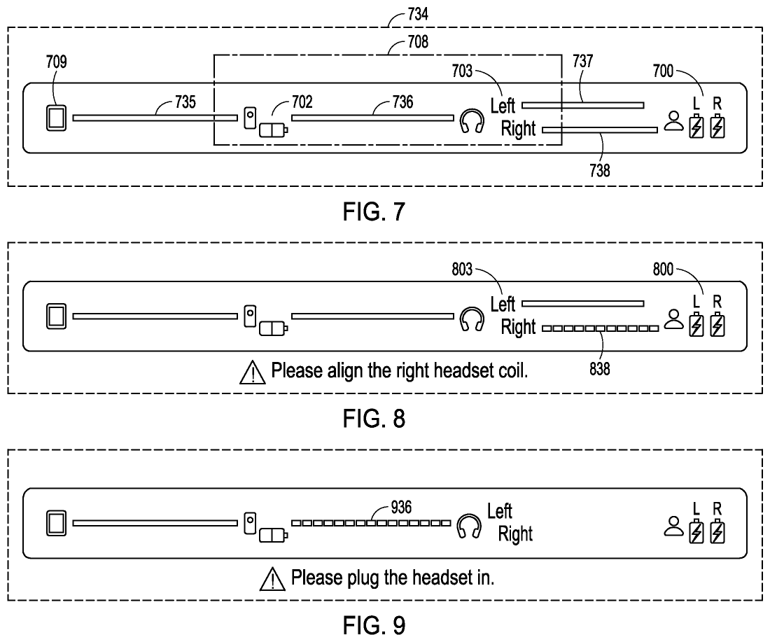

FIG. 7 illustrates, by way of example and not limitation, a display with representations for a multi-device neuromodulation system. The system may include a number of different system elements configured to communicate with each other via communication links, and may include a display screen 734. At least some of the system elements may be powered by a battery (e.g., rechargeable battery), and at least some of the system elements may be programmed. At least one or more of the system elements may be configured to transfer power to other one(s) of the system elements. For example, the multi-device neuromodulation system may include at least two implantable devices 700, an external system 735 configured to communicate with each of the at least two implantable devices 700, and a programmer 709 configured to communicate with the external system 708. The programmer may include a user interface where the user interface includes a display, with the display screen 734 presented on the display. The programmer may also include a processor configured to provide information on the display, including the multi-device neuromodulation system. The external system 708 may include multiple devices, such as an external device 702 operably connected to a headset 703. The representation for the illustrated system includes representations (or icons) for two implantable devices, an external system used to communicate and/or charge the implantable devices, and a programmer. The headset may include coils (e.g., Tx coils) used to communicate with and transfer power to the implanted devices.

The illustrated representation for the system includes representations for links 735, 736, 737 and 738 between devices for communication and/or power transfer. The links to the implantable devices may be wireless links that allow the external device to communicate and/or transfer power to the implantable devices. The representation for the link between the programmer and the external system may be a wired (e.g., cable) or wireless link. Similarly, the link between the external device and the headset may be a wired (e.g., cable) or wireless link. If these communication links are broken or not in appropriate communication range the user may not be able to retrieve or change therapy information on the device and/or may not be able to be used to transfer power. The representation for the link may be designed to provide information about the state of the communication and/or power link. For example, color (e.g., green or another color) and/or line type (e.g., solid line) may be used to indicate that a good link is established between system components. Alternatively, or additionally, labels may be used to provide information about the link state. Also, at least some of the system elements that are powered by a battery may provide an indication of their charge state using color, symbols such as a full battery or partially full battery, and/or labels.

FIG. 8 illustrates, by way of example and not limitation, by way of example and not limitation, representations in a display of FIG. 7, indicative of a poor link between the headset icon and the right implantable device icon. The poor link 838 may be communicated using a different color (e.g., gray or other color) and/or different line type (e.g., dotted line). In this representation the communication between right Tx coil in the headset 803 and the right implant 800 is not in range of fully aligned. The system may be able to determine that the poor link is caused by (or likely caused by) poor alignment of the Tx coil in the headset, and may be configured to notify the user of the failure in the link and informed to perform alignment. Should the system determine that the charge state is low, the system may notify the use to charge the device(s) with the low charge state.

FIG. 9 illustrates, by way of example and not limitation, representations in a display of FIG. 7, indicative of a poor link between devices in the external system. Similar to FIG. 8, the poor link 936 may be communicated using a different color (e.g., gray or other color) and/or different line type (e.g., dotted line).

For example, the poor link may be between the external device and the headset, and may be caused by the cable not being fully plugged into the external device. The system may be able to determine that the poor link is caused by (or likely caused by) poor alignment of the Tx coil in the headset, and may be configured to prompt the user to take action (e.g., prompt the user to plug in their headset).

Each of the implantable devices may be programmed with a stimulation configuration. The stimulation configuration may include an electrode configuration indicating which electrodes are active and which electrodes are inactive, and may further be configured to determine which active electrode(s) are anodic and which electrode(s) are cathodic. The stimulation configuration may also provide stimulation waveform configurations, such as amplitude. Other stimulation waveform configurations may include frequency and pulse width, by way of example and not limitation. In some embodiments, the same stimulation waveform configuration is delivered to all of the active anode(s) and active cathode(s). In some embodiments, each electrode may be independently programmed with a stimulation waveform configuration.

The system design may limit which stimulation configurations are allowed. The system may be configured to display a current selection of a stimulation configuration, provide an indication if the stimulation configuration is allowable, and provide guidance for changing the stimulation configuration into an allowable stimulation configuration. For example, the stimulation configuration may include a user-selected electrode configuration and a user-selected waveform configuration (which may be limited to a user-selected amplitude configuration). The processor may be configured to guide the user to select at least one anode electrode and at least one cathode electrode. The processor may be configured to guide the user to select allowable amplitudes for a selected electrode configuration.

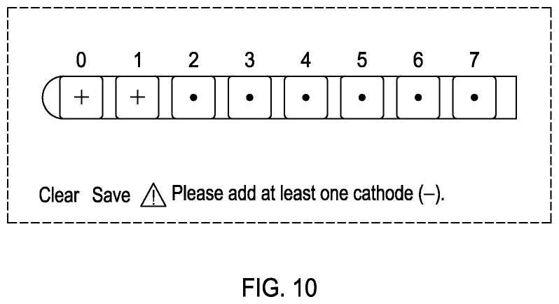

FIG. 10 illustrates, by way of example and not limitation, representations in a display indicative of an allowable electrode configuration. In the illustrated example, the system requires at least one anode (+) and one cathode (−) for stimulation to function. The processor may determine the invalid configuration as the user manipulates the user interface, may disable to save button to prevent the invalid configuration from being saved, prompt the user to take the specific action needed to provide a valid configuration. In the illustrated embodiment, the processor may be configured to guide the user to configure at least one cathode (−) in the electrode configuration. Similarly, if the user did not select an anode, the processor may be configured to guide the user to configure at least one anode (+).

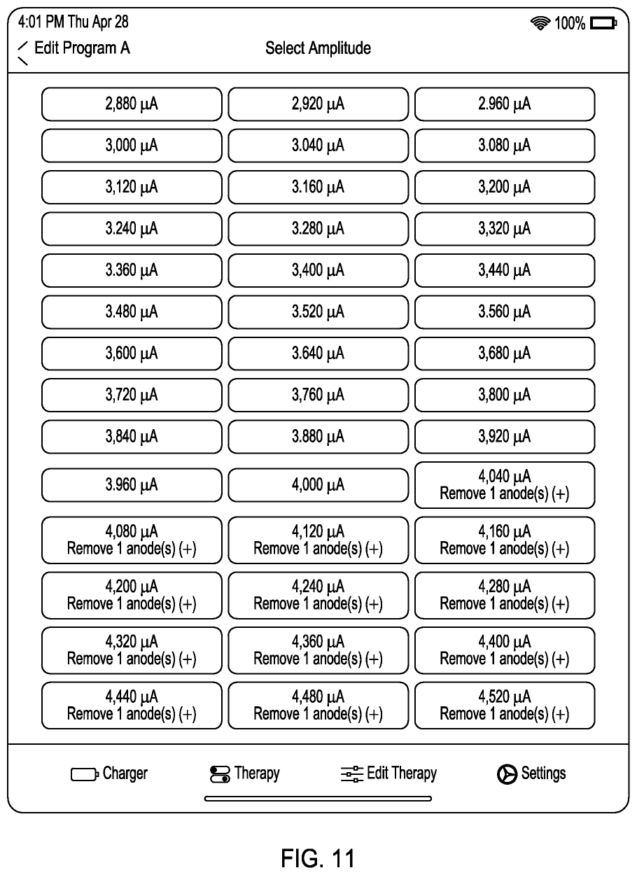

FIG. 11 illustrates, by way of example and not limitation, representations in a display indicative of an allowable waveform configuration for selectable electrode configurations. The system may only support certain amplitudes for certain electrode configurations. The processor may be configured to use the display to inform the user of viable electrode and amplitude configurations. Should the user want to increase an amplitude to a higher level they are informed of the acceptable electrode configurations for the amplitude or are informed of the required changes to electrode configuration are required to make the adjustment. The allowable waveform configurations may be displayed differently than the waveform configurations that are not allowable. For example, different colors (e.g., hue, value and/or intensity), different line types and/or thicknesses, and the like may be used to distinguish whether a configuration is allowable or not allowable. The system may disable amplitudes that are invalid for a selected electrode configuration. Similar displays may be created for different allowable combinations of two or more configurations from both an electrode configuration and a waveform configuration, and/or two or more configurations from a waveform configuration, where the selection of one configuration determines which other configurations are enabled for selection or disabled for selection.

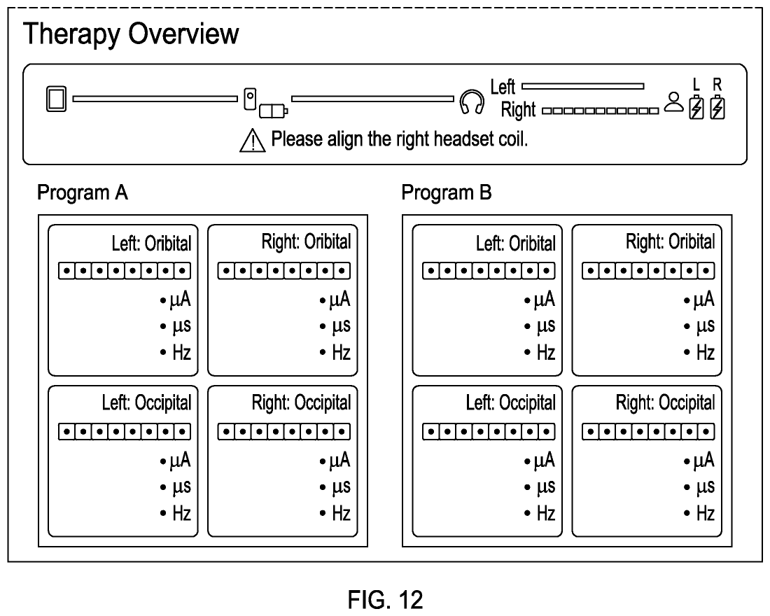

FIG. 12 illustrates, by way of example and not limitation, representations in a display indicative of different regions in the body. The display screen illustrates two different programs (Program A and Program). Each displayed program may include electrode configuration and waveform configuration information for different regions of the body (e.g., a left orbital region, a right orbital region, a left occipital region and a right occipital region). Each of the different regions represented in the display may include an electrode configuration, an amplitude, a pulse width and a frequency. The electrodes are illustrated with “dots” indicating no programmed polarity. Once programmed, the polarity may be identified by displaying “+” or “−”, or by displaying other nomenclature for cathodic and anodic electrodes. The programmer may be configured for use to program the at least two implantable devices by associating the different stimulation configurations with the different regions of the body, and receive programming inputs for the different stimulation configurations associated with the different regions of the body. The user interface may a display that includes identifiers for each of the different regions of the body and includes the different stimulation configurations associated with the different regions. In the illustrated embodiment, for example, the system is configured to preset programming inputs that correspond to nerve location and/or body regions (quadrants). This abstracts the user from what actual device is needed to be programmed.

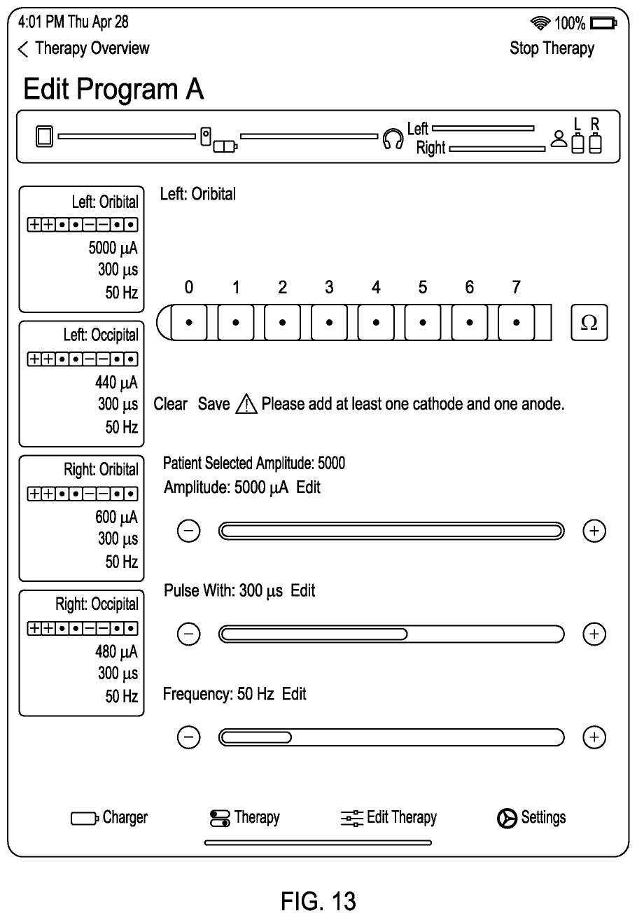

FIG. 13 illustrates, by way of example and not limitation, a screen display for editing a user-selected program in FIG. 12. Different regions of the body may be displayed. The display also has an edit program region which may be used to edit the stimulation configuration for a user-selected one of the programs. The displayed regions of the body may include identifiers for the current stimulation configuration (e.g., electrode configuration, amplitude, pulse width and frequency). A user input may be used to select one of the different regions of the body for editing. Upon selection, the user interface may display component(s) of the stimulation configuration for editing. For example, the display may include user-adjustable representation(s) corresponding to the electrode configuration(s), and may further include user-adjustable representation(s) corresponding to an amplitude, pulse width and frequency of the stimulation delivered for the program.

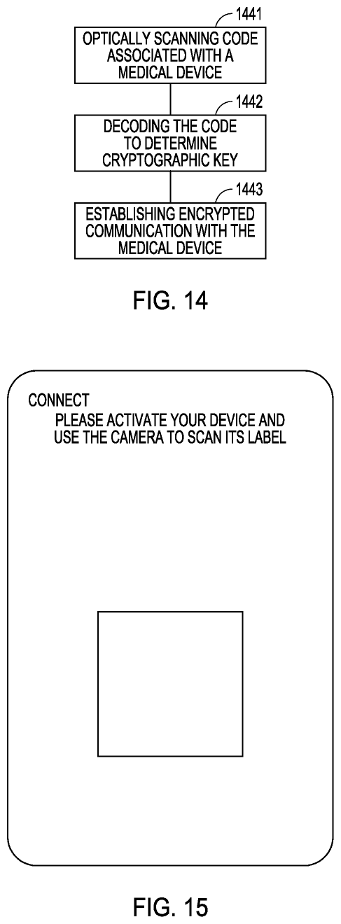

The present subject matter also provides an improved method for establishing secure communication between devices within the system. FIG. 14 illustrates, by way of example and not limitation, a method for using an optically-scanned code to establish encrypted communication. The programming of the medical device(s) may use encrypted communication channels. Various embodiments of the present subject matter may use an optically-scannable code to provide a programmer device with a cryptographic key. The code may be a two-dimensional barcode for encoding alphanumeric information, and the alphanumeric information includes the cryptographic key. For example, the optically-scannable code may be a Quick Response (QR) code. The code may be on packaging or otherwise associated with the medical device(s) so that the camera that is used to scan the code is local to the medical device(s). The method may include optically scanning a code associated with a medical device 1441, wherein a cryptographic key for the medical device is encoded within the code, decoding the code to determine the cryptographic key 1442, and establishing encrypted communication with the medical device using the cryptographic key 1443. A first device, such as an external device with a camera, performs the optical scanning and the decoding of the code to determine the cryptographic key. The first device may use the cryptographic key to enable encrypted communication using the cryptographic key to encode a first string of bytes to provide an encoded first string of bytes, and sending the encoded first string of bytes to the medical device. The medical device may use a first private key stored in the medical device to decode the encoded first string of bytes to provide a decoded first string of bytes, and may use the decoded first string of bytes to encode a second string of byte to provide an encoded second string of bytes, and sends the encoded second string of bytes to the first device. Both the first device and the medical device may session keys based on the encoded first string of bytes and the encoded second string of bytes, and may use the generated session keys to provide the encrypted communication. The same device used to optically scan the code may be used to decode the code and to establish the encrypted communication. In some embodiments, different devices, that already communicate within a secure network, may be used to scan the code, decode the code the establish encrypted communication with the medical device.

FIG. 15 illustrates, by way of example and not limitation, a display of a device used to obtain a cryptographic key for use to establish encrypted communication. For example, the device may be designed with an integrated camera. The device may be, by way of example and not limitation, a phone, a tablet or a mobile computer. The device may be a programmer. When the programmer is initiated to first communicate with the medical device, the programmer may request the user to use the integrated camera. The device may be positioned over the code, or the code may be moved within a field of view of the device's camera. The relative positions of the code and the camera are adjusted until code is within the rectangle of the camera's user display.

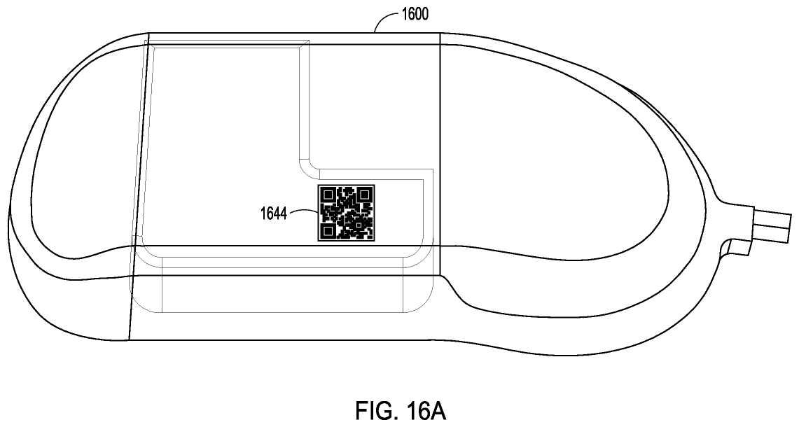

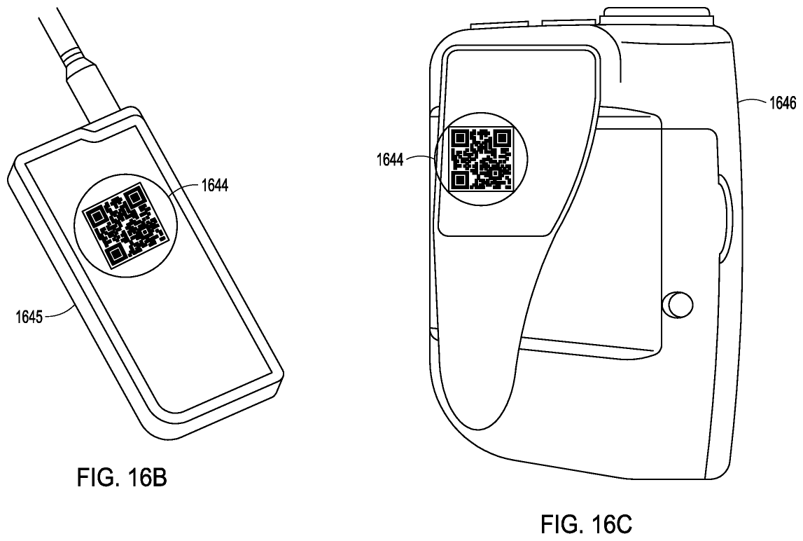

FIG. 16A illustrates, by way of example and not limitation, a QR code 1644 on the implantable device 1600 for use in obtaining a cryptographic key to establish encrypted communication with the implantable device. FIG. 16B illustrates, by way of example and not limitation, a QR code 1644 on a patient programmer 1645 for use in obtained a cryptographic key for use in establishing encrypted communication with the patient programmer. FIG. 16C illustrates, by way of example and not limitation, a QR code 1644 on an external trial stimulator for use in obtained a cryptographic key for use in establishing encrypted communication with the external trial stimulator 1646. The system (e.g., programmer) may establish encrypted communication with at least one medical device such as an implantable device 1600, a patient programmer 1645 and/or an external trial stimulator 1646. Other embodiments may provide the code in literature provided with the medical device(s), or packing of the medical device(s). Thus, in order for the system to securely communicate with the illustrated medical devices, at least some components of the system must be near the medical device in order to scan the code. This proximity provides a level of security for exchanging a cryptographic key.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system, comprising:
at least two implantable devices;
an external system configured to communicate with each of the at least two implantable devices;
a programmer configured to communicate with the external system, the programmer including:
a user interface, the user interface including a display; and
a processor configured to provide on the display:
implantable device icons corresponding to each of the at least two implantable devices,
an external system icon corresponding to the external system, and
wireless link representations corresponding to wireless links between the external system and each of the at least two implantable devices, wherein the system is configured to use the wireless links for at least one of communication or power transfer.

2. The system of claim 1, wherein the wireless representations provide a status indicator for a current status for each of the wireless links.

3. The system of claim 2, wherein the processor is configured to provide on the display a suggested action to remedy a problem with the wireless status.

4. The system of claim 1, wherein the external system includes a headset, and the headset includes at least two external coils, wherein the external system is configured to provide wireless links, wherein the wireless links include a first link between a first one of the external coils and a first one of the implantable devices, and a second link between a second one of the external coils and a second one of the implantable devices, wherein the wireless link representations provide status indicators for corresponding wireless links between each of the at least two external coils and the at least two implantable devices.

5. The system of claim 4, wherein the processor is configured to provide on the display a suggestion to align a specific one of the external coils when the status indictor for the wireless link corresponding to the specific one of the coils indicates misalignment.

6. The system of claim 4, wherein the external system includes an external device configured to be connected to the headset via a cable, wherein the processor is configured to provide on the display a cable representation corresponding to a connection of the cable between the headset and the external device, and the processor configured to provide on the display, when a status indicator for the cable representation indicates a failed connection between the headset and the external device, a suggestion to remedy the failed connection.

7. The system of claim 1, wherein the processor is further configured to provide on the display a programmer icon corresponding to the programmer, wherein the communication link representations further include a communication link representation corresponding to a communication link between the programmer icon and the external system icon.

8. The system of claim 1, wherein each of the implantable devices include a rechargeable battery, and the processor is further configured to provide on the display charge state representations for the rechargeable battery in each of the implantable devices.

9. The system of claim 1, wherein each of the at least two implantable devices are configured to be programmed with a stimulation configuration, wherein the processor is configured to guide a user with acceptable programming inputs when programming the stimulation configuration.

10. The system of claim 9, wherein the stimulation configuration includes an electrode configuration and an amplitude configuration.

11. The system of claim 9, wherein the processor is configured to guide the user to select at least one anode electrode and at least one cathode electrode.

12. The system of claim 9, wherein the processor is configured to guide the user to select allowable amplitudes for a selected electrode configuration.

13. A system, comprising:
at least two implantable devices, each of the at least two implantable devices being configured to deliver stimulation to different regions of a body according to different stimulation configurations;
an external system configured to communicate with each of the at least two implantable devices; and
a programmer configured to communicate with the external system, the programmer being configured for use to program the at least two implantable devices by associating the different stimulation configurations with the different regions of the body, and receiving programming inputs for the different stimulation configurations associated with the different regions of the body, wherein the programmer includes a user interface, and the user interface includes a display that includes identifiers for each of the different regions of the body and includes the different stimulation configurations associated with the different regions.

14. The system of claim 13, wherein the different regions include a left orbital region, a right orbital region, a left occipital region and a right occipital region.

15. The system of claim 13, wherein the display includes, for each of the different regions, an electrode configuration, an amplitude, a pulse width and a frequency.

16. A method, comprising:
optically scanning a code associated with a medical device, wherein a cryptographic key for the medical device is encoded within the code;
decoding the code to determine the cryptographic key; and
establishing encrypted communication with the medical device using the cryptographic key.

17. The method of claim 16, wherein the code is a two-dimensional barcode for encoding alphanumeric information, and the alphanumeric information includes the cryptographic key.

18. The method of claim 17, wherein the code is a Quick Response (QR) code.

19. The method of claim 16, wherein:
a first device performs the optical scanning and the decoding of the code to determine the cryptographic key, the first device uses the cryptographic key to enable encrypted communication using the cryptographic key to encode a first string of bytes to provide an encoded first string of bytes, and sending the encoded first string of bytes to the medical device;
the medical device uses a first private key stored in the medical device to decode the encoded first string of bytes to provide a decoded first string of bytes, and uses the decoded first string of bytes to encode a second string of byte to provide an encoded second string of bytes, and sends the encoded second string of bytes to the first device; and
both the first device and the medical device generate session keys based on the encoded first string of bytes and the encoded second string of bytes, and use the generated session keys to provide the encrypted communication.

20. The method of claim 16, wherein
the optically scanning the code includes using a camera in a first device to optically scan the code;
the decoding the code includes using the first device to decode the code; and
the establishing encrypted communication includes establishing encrypted communication between the first device and the medical device.

21. The method of claim 16, wherein a programmer is used to establish encrypted communication with the medical device, and wherein the medical device includes an implantable stimulator, an external trial stimulator or a charger.

22. A method performed in a system having at least two implantable devices, an external system configured to communicate with each of the at least two implantable devices, and a programmer configured to communicate with the external system, wherein the method includes:
displaying a representation of the system on the display; and
performing at least one of:
determining a link status for at least links between the external system and the implantable devices, displaying the link status within the displayed representation of the system; or
determining a status of rechargeable batteries, and displaying the rechargeable battery status within the displayed representation of the system.

23. The method of claim 22, further comprising suggesting remedial action for the determined link status or the determined status of rechargeable batteries.

24. The method of claim 22, further comprising guiding selection of an allowable stimulation configuration for the system.
